(19) United States
(12) Patent Application Publication
(10) Pub. No.: US 2009/0007299 A1
(43) Pub. Date: Jan. 1, 2009

(54) FORAGE BERMUDAGRASS CULTIVAR

(75) Inventors: YANQI WU, Stillwater, OK (US); CHARLES M. TALIAFERRO, Stillwater, OK (US); RICK KOCHENOWER, Guymon, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: THE BOARD OF REGENTS FOR OKLAHOMA STATE UNIVERSITY, Stillwater, OK (US)

(21) Appl. No.: 12/050,292

(22) Filed: Mar. 18, 2008

Related U.S. Application Data
(60) Provisional application No. 60/946,516, filed on Jun. 27, 2007.

(51) Int. Cl. A01H 5/00

(52) U.S. Cl. 800/298

(57) ABSTRACT

“Goodwell” is a hybrid forage bermudagrass. It has larger stems and wider leaves, and produces a more dense sod compared to “hay type” varieties. The stem size and leaf width is much greater compared to “grazing type” varieties while the sod density is somewhat less compared to the grazing type varieties.

### CROSS REFERENCE TO RELATED APPLICATIONS

This application claims the benefit of prior filed U.S. Provisional Patent Application No. 60/946,516 entitled “FORAGE BERMUDAGRASS CULTIVAR,” filed Jun. 27, 2007, the contents of which are hereby incorporated by reference.

### BACKGROUND

“GOODWELL” is a new forage bermudagrass. This cultivar is also referred to herein by its experimental designation LCB 84X 16-66.

LCB 84X 16-66 is an F₁ hybrid from the cross 74X 12-11×74X 12-12, made in 1984. The 74X 12-11 and 74X 12-12 parents were F₁ hybrids from the crosses A9959×SS-28 and SS-16×Colorado, respectively. The SS-28 and SS-16 parents were F₁ hybrids from the crosses S-16×A9945 and S-16×9958, respectively. The S-16 parent was an F₁ hybrid from the cross A8800×A10421. A9945 (PI 206427), A9958 (PI 251809), A9959 (PI 253302), Colorado, and A8800 (PI 269370) are clonal accessions from Turkey, Italy, Yugoslavia, Colorado, and Afghanistan, respectively. Several hundred F₁ hybrid plants from many crosses, including 74X 12-11×74X 12-12, were initially screened in a planting at Lake Carl Blackwell during the period 1985 through 1987. The LCB 84X 16-66 hybrid was one of several plants selected from this nursery for further evaluation.

### SUMMARY OF THE INVENTION

GOODWELL is an asexually reproducing forage bermudagrass. It has been evaluated for forage yield in several trials, performing favorably relative to standard varieties. Data from field trials and observational plantings indicate GOODWELL to be well-adapted to production under irrigation in the Oklahoma panhandle and adjacent areas of the High Plains. Accordingly, the targeted use of the variety is in this region under irrigation. GOODWELL has generally initiated spring growth slightly earlier than other common varieties, suggesting an edge in winter hardiness. GOODWELL also exhibits larger stems and wider leaves compared to other commercial available varieties. GOODWELL exhibits greater sod density that “hay type” varieties and taller growth than “grazing type” varieties. These are favorable characteristics for certain situations.

### BRIEF DESCRIPTION OF THE DRAWINGS

FIG. 1 is a photograph of a 9-10 week old growth of GOODWELL.

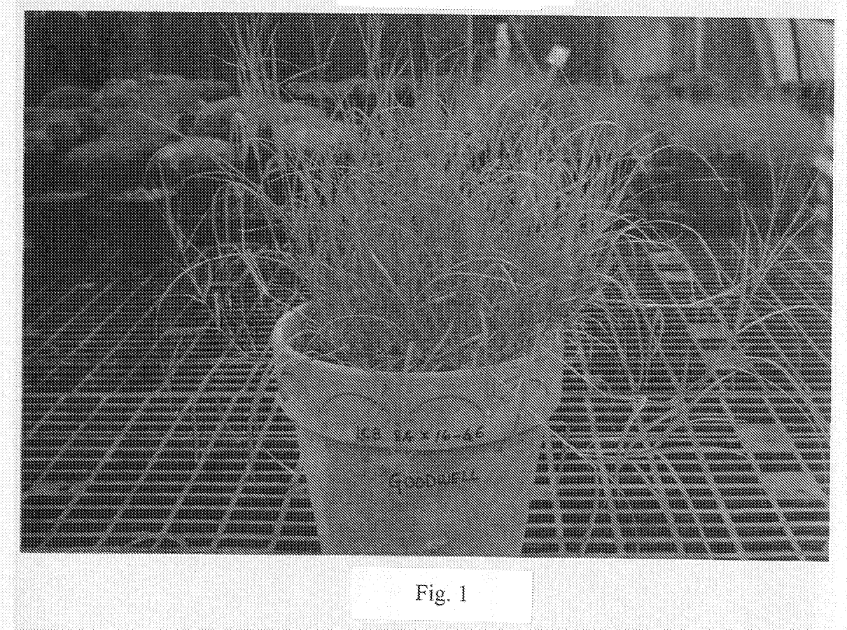

FIG. 2 is a comparative leaf image of MIDLAND 99 (left), GOODWELL (center), and GREENFIELD (right) varieties.

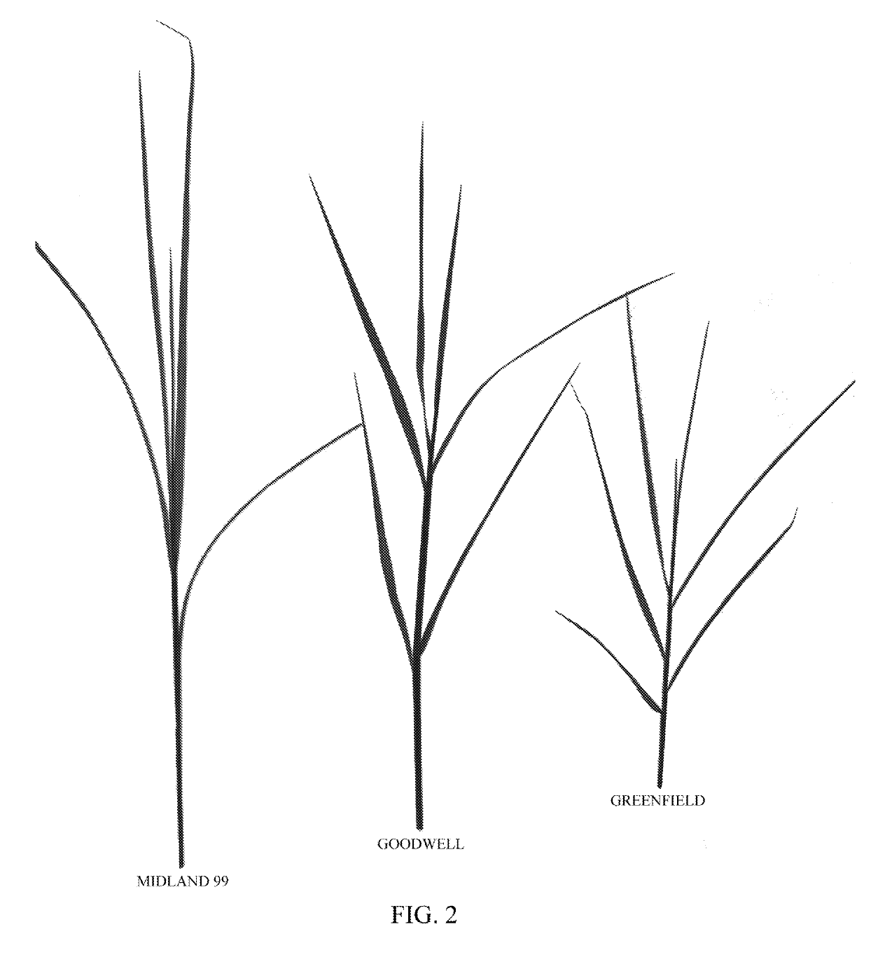

FIG. 3 is another comparative leaf image of MIDLAND 99 (left), GOODWELL (center), and GREENFIELD (right) varieties.

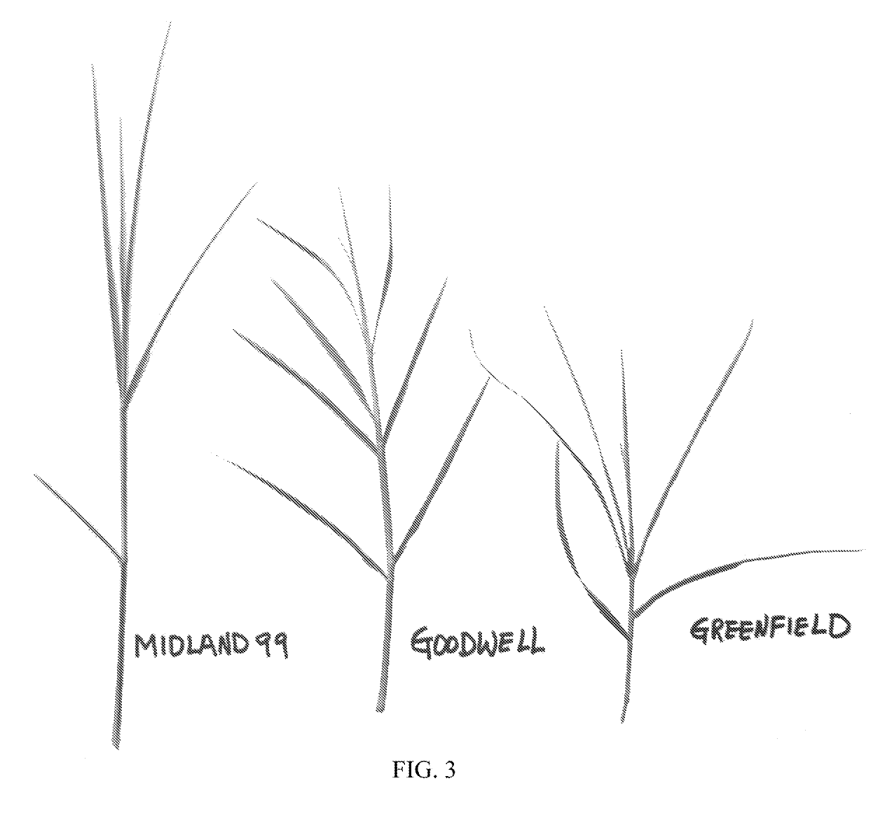

FIG. 4 is an image of two typical GOODWELL inflorescences, one with four racemes and the other with five.

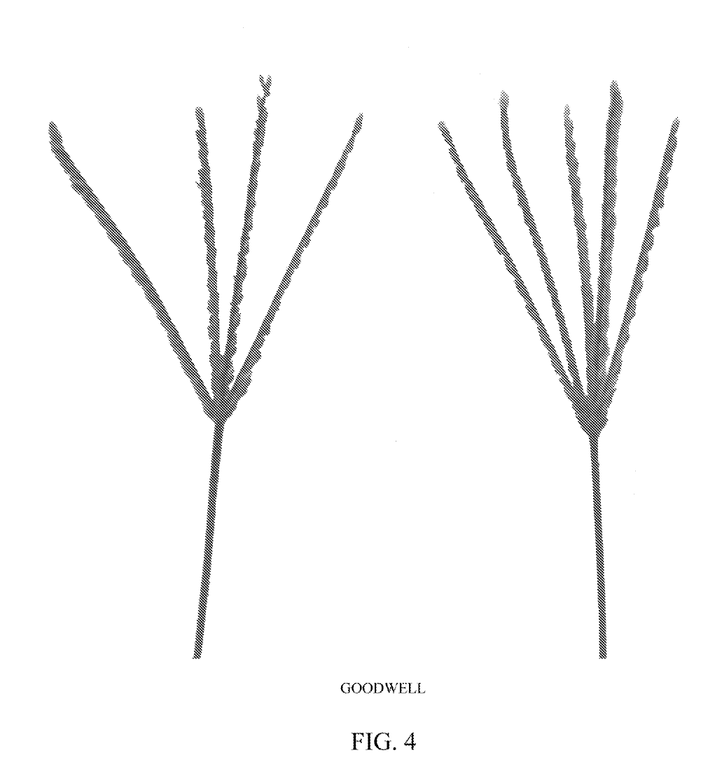

FIG. 5 is a close-up image of the typical inflorescence of a sample GOODWELL plant showing five racemes.

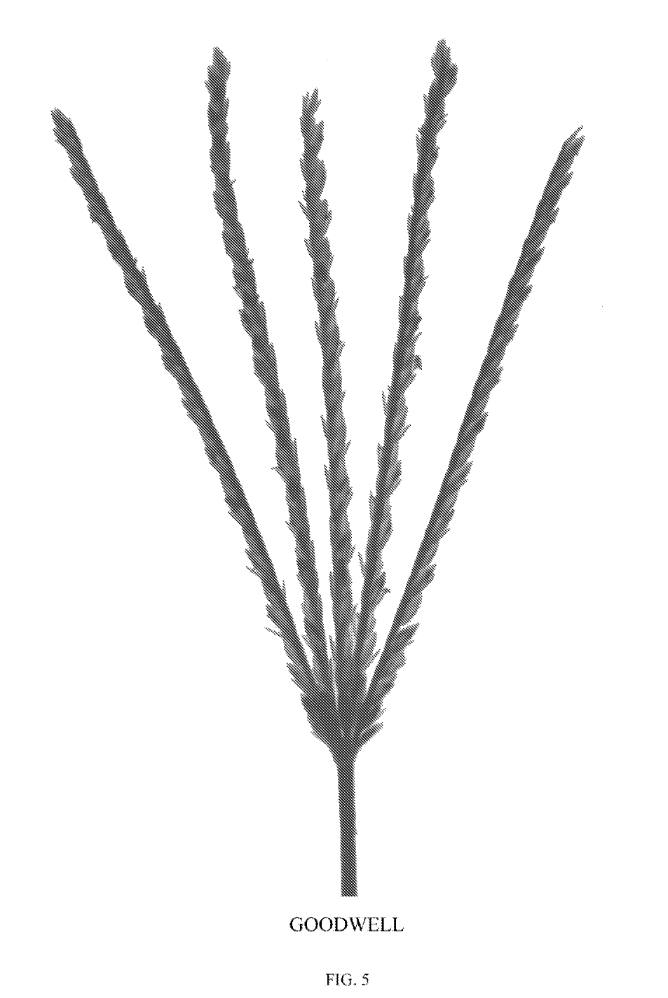

FIG. 6 is an image of GOODWELL growing in a field trial next to A-12245, Sey Greenfield, Ozark, World Feeder, Vaughn's #1, and Midland 99.

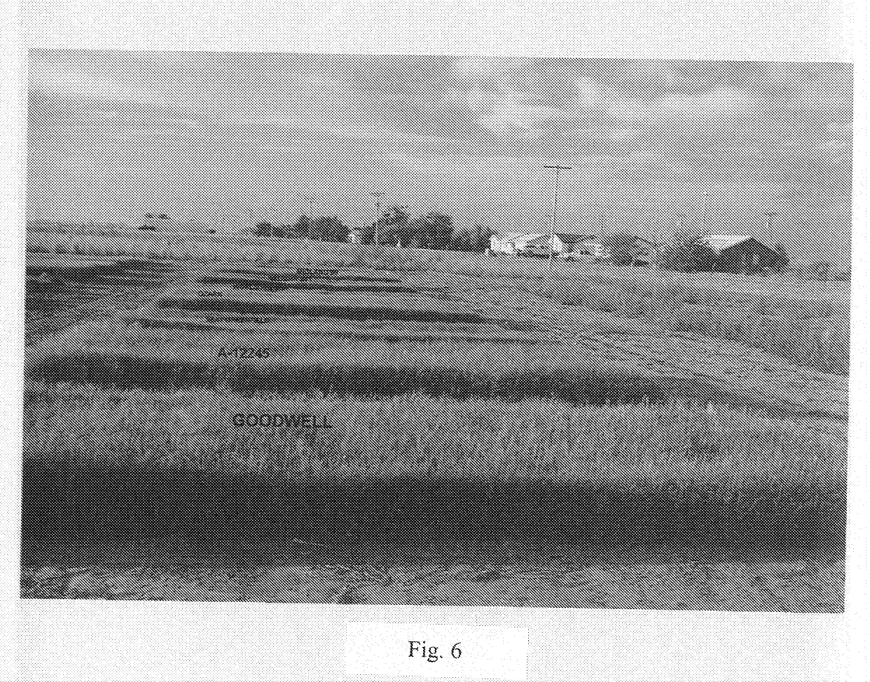

### DETAILED BOTANICAL DESCRIPTION

Goodwell, a bermudagrass, Cynodon dactylon (L.) Pers. (LCB 84X 16-66) is an F₁ hybrid from the cross 74X 12-11×74X 12-12, made in 1984. The 74X 12-11 and 74X 12-12 parents were F₁ hybrids from the crosses A9959×SS-28 and SS-16×Colorado, respectively. The SS-28 and SS-16 parents were F₁ hybrids from the crosses S-16×A9945 and S-16×9958, respectively. The S-16 parent was an F₁ hybrid from the cross A8800×A10421. A9945 (PI 206427), A9958 (PI 251809), A9959 (PI 253302), Colorado, and A8800 (PI 269370) are clonal accessions from Turkey, Italy, Yugoslavia, Colorado, and Afghanistan, respectively. Several hundred F₁ hybrid plants from many crosses, including 74X 12-11×74X 12-12, were initially screened in a planting at Lake Carl Blackwell during the period 1985 through 1987. The LCB 84X 16-66 hybrid was one of several plants selected from this nursery for further evaluation.

LCB 84X 16-66 has larger stems and wider leaves, and produces a more dense sod compared to “hay type” varieties like Midland, Midland 99, and Tifton 44, LCB 84X 16-66. It is typically lower growing (less tall) as well. The stem size and leaf width of LCB 84X 16-66 is much greater compared to “grazing type” varieties like Greenfield and World Feeder. The height of LCB 84X 16-66 is typically greater, while the sod density is somewhat less compared to the grazing type varieties. LCB 84X 16-66 produces many large, fleshy rhizomes that contribute to lateral spread during establishment. Lateral spread is also achieved by growth of stolons.

LCB 84X 16-66 is highly infertile, producing only very few seed when grown in the presence of an effective pollinator. Consequently, it must be propagated asexually. Chromosome number has not yet been determined. It is likely a tetraploid with 2n=4x=36 chromosomes.

LCB 84X 16-66 is propagated by conventional sprigging. It produces vigorous underground rhizomes and crown buds that function as vegetative propagules. Though not evaluated in a controlled experiment, its establishment characteristics (sprig viability, rate-of-spread) appear to be at least as good, and possibly superior to those of Midland, Midland 99, and Tifton 44. It's rate-of-spread probably is not as rapid as some of the aggressive “common” type varieties like Greenfield and World Feeder. A sprig planting rate of 30 or more bushels/acre, combined with good weed control and fertility management is recommended to hasten establishment.

Biomass production has been evaluated in trials at Haskell, Okla. (Eastern Research Station), Chickasha, Okla. (South Central Research Station), Goodwell, Okla. (Oklahoma Panhandle Research & Extension Center), and Mound Valley, Kans. (Kansas State University Southeast Agricultural Research Center). Biomass performance of LCB 84X 16-66 relative to standard varieties has been best in irrigated (˜6 acre inches/month during growing season) tests at Goodwell, Okla. In Tests 97-1 and 2003-1 at Goodwell, LCB 84 X 16-66 produced significantly (P>0.05) more biomass than all standard varieties except Ozark (Tables 1 and 2). In tests at Haskell [Test 98-1 (Table 3); Test 2001-1 (Table 4)] and Chickasha [Test 98-2 (Table 5); Test 2001-2 (Table 6)] LCB 84X 16-66 produced less biomass than the best hay type varieties (Midland 99, Ozark, Tifton 44), but more biomass than the grazing type varieties (e.g. Greenfield). At Mound Valley, Kans., its biomass yield was not significantly greater than the yields of Ozark and Hardie, but significantly greater than Tifton 44, Midland, Greenfield, and World Feeder (Table 7).

Near Infrared Reflectance Spectroscopy predicted values for neutral detergent fiber, acid detergent fiber, acid detergent lignin, crude protein and digestible dry matter for samples collected during 1986-87 suggest its nutritional value is at least equal to that of Tifton 44 and Midland (Table 8).

There have been no reports or observations of any unusual and/or severe insect or disease problems with LCB 84X 16-66. Leaf disease on LCB 84X 16-66 has been minimal when other bermudagrass varieties showed severe infections. The principal leaf spotting disease of bermudagrass caused by Bipolaris cynodontis is a major cause of performance (stand and biomass) decline in humid environments.

LCB 84X 16-66 has maintained good stands at all locations where it has been tested (Chickasha, Haskell, Goodwell, Stillwater, and Mound Valley, Kans.). These results indicate its good adaptation to at least the bermudagrass growing area north of central Oklahoma. The consistent good performance of LCB 84X 16-66 at Goodwell appears to be related, at least in part, to good freeze tolerance. It has showed no winter injury at Goodwell during the period 1998 through 2006. In the Goodwell tests, it has consistently been the earliest, or among the earliest, varieties to initiate spring growth. It also demonstrated excellent winter survival and spring greenup at Mound Valley, Kans.

LCB 84X 16-66 was tested under pivot irrigation near Goodwell, Okla. for the past 3 years by a local farmer under a memorandum-of-agreement. An initial 60 acres was established in 2004 and the remaining 60 acres under the irrigation circle was established in 2005. Establishment was good for both plantings, but better for the 2004 planting compared to the 2005 planting. Stocker cattle grazed the initially established 60 acres during the 2005 growing season. The farmer reported the following: an initial stocking rate of 5.28 head/acre (317 animals on 60 acres); 100 animals weighed off at 95 days gained 1.78 lbs./day; season-long total gain from all animals ˜53,000 pounds, with an average daily gain of ˜1.5 pounds. Gains of stocker cattle grazing the 120 acres during the 2006 growing season were not good (≦˜0.5 lbs./day), though carrying capacity continued very high. Reasons for the poor performance in 2006 are unknown, but possible contributing factors were: 1) relatively poor quality of the stocker animals, and 2) excessive maturity of the bermudagrass and consequent low nutritional value.

The farmer has over-seeded the LCB 84X 16-66 with small grains each fall, beginning in fall 2004. This has been easily accomplished and highly successful. Growers of World Feeder bermudagrass in the same region have had difficulty interseeding cool-season species, apparently due to its greater sod density. The farmer highly values ability to easily inter-seed cool season species into LCB 84X 16-66.

The farmer also grows cool-season perennial grasses (bromegrass, orchardgrass) under irrigation circles. He believes the warm-season bermudagrass enhances his stocker grazing forage system. Additionally, he believes that the bermudagrass offers significant opportunity as hay crop in the High Plains region under irrigation.

Data from field trials and observational plantings indicate LCB 84X 16-66 to be well-adapted to production under irrigation in the Oklahoma panhandle and adjacent areas of the High Plains. Accordingly, the targeted use of the variety is in this region under irrigation. It has produced lower biomass yields than Tifton 44 and Midland 99 in tests at Chickasha and Haskell. However, even in bermudagrass growing areas outside the High Plains, LCB 84X 16-66 may have some advantage over varieties like Midland, Midland 99, and Tifton 44 relative to faster establishment and a more dense sod that better resists weed encroachment.

For the High Plains region, the closest competitor of LCB 84X 16-66 among commercial bermudagrass varieties is Ozark. Both combine excellent winter hardiness and very high biomass production capability in this region. LCB 84X 16-66 has generally initiated spring growth slightly earlier than Ozark at Goodwell, suggesting an edge in winter hardiness. The greater sod density of LCB 84X 16-66 compared to Ozark may be desirable when used for grazing at high stocking levels (more resistant to trampling injury). Conversely, the generally taller growth of Ozark compared to LCB 84X 16-66 may make it more attractive to producers when the intended use is primarily or solely for hay production. The greater sod density of LCB 84X 16-66 compared to Ozark should also make it more resistant to weed encroachment.

TABLE 1
Forage yields (tons dry matter/acre) of commercial and experimental
bermudagrass varieties in Test 1997-1, Oklahoma Panhandle
Research and Extension Center, Goodwell OK. 1998-2003.

| | Harvest Year | | | | | | |
|---|---|---|---|---|---|---|---|
| | 1998 | 1999 | 2000 | 2001 | 2002 | 2003 | 6-yr |
| Variety | 4-cuts | 4-cuts | 4-cuts | 4-cuts | 4-cuts | 4-cuts | Mean |

Commercial Varieties - Available for Farm Use

| | | | | | | | |
|---|---|---|---|---|---|---|---|
| Ozark | 11.84* | 8.00* | 9.94* | 14.53* | 13.25* | 15.10* | 12.11* |
| Midland 99 | 10.16 | 7.60 | 8.53 | 13.00 | 12.86 | 13.48 | 10.94 |
| Hardie | 12.99** | 8.03* | 7.98 | 13.21 | 10.94 | 11.82 | 10.83 |
| Midland | 8.64 | 5.32 | 7.47 | 11.85 | 12.06 | 12.66 | 9.66 |
| Guymon | 9.65 | 4.49 | 5.51 | 11.16 | 11.60 | 12.20 | 9.23 |
| Tifton 44 | 9.23 | 5.48 | 6.98 | 10.39 | 11.00 | 12.34 | 9.10 |
| Wrangler | 10.00 | 4.59 | 5.55 | 10.25 | 11.76 | 11.66 | 8.97 |
| Quickstand | 9.86 | 5.77 | 6.04 | 10.76 | 9.17 | 10.74 | 8.72 |
| Greenfield | 8.91 | 4.18 | 5.24 | 9.73 | 10.86 | 13.15 | 8.68 |

Experimental Varieties - Not Available for Farm Use

| | | | | | | | |
|---|---|---|---|---|---|---|---|
| ERS 94X 2-8 | 11.65* | 8.99* | 10.29** | 13.10 | 14.80** | 15.97** | 12.46** |
| LCB 84X 16-66 | 11.93* | 8.60* | 8.86 | 16.07* | 14.51* | 14.49* | 12.41* |
| LCB 84X 19-16 | 11.59* | 9.74** | 8.51 | 16.24** | 13.89* | 14.02* | 12.33* |
| CD 90160 | 11.75* | 6.85 | 7.56 | 13.30 | 13.67* | 13.35 | 11.08 |
| ERS 94X 13-9 | 9.48 | 6.45 | 7.95 | 13.00 | 13.30* | 15.53* | 10.95 |
| SCRS-C | 10.61 | 5.85 | 7.39 | 11.43 | 10.98 | 11.93 | 9.70 |
| ERS 94X 5-12 | 9.23 | 5.57 | 6.31 | 11.10 | 12.29 | 13.57 | 9.66 |
| ERS-C | 8.82 | 4.76 | 5.50 | 9.75 | 11.00 | 13.14 | 8.83 |
| A-12199 | 7.61 | 5.03 | 5.20 | 9.95 | 9.94 | 11.53 | 8.21 |
| ERS 94X 6-13 | 8.08 | 5.07 | 6.13 | 7.69 | 9.04 | 11.28 | 7.88 |
| Mean | 10.11 | 6.33 | 7.21 | 11.91 | 11.94 | 13.05 | 10.09 |
| CV (%) | 16 | 20 | 11 | 15 | 13 | 13 | 15 |
| 5% LSD | 2.34 | 1.77 | 1.09 | 2.60 | 2.14 | 2.39 | 0.85 |

**Highest numerical value in column.
*Not significantly different from the highest numerical value in the column based on 5% LSD.

TABLE 2
Forage yields of bermudagrass varieties in Test 2003-1,
Oklahoma Panhandle Research & Extension Center,
Goodwell, OK. 2004-2006.

| | Year | | | |
|---|---|---|---|---|
| | 2004 | 2005 | 2006 | |
| | 3-Cuts | 4-Cuts | 3-Cuts | |
| Variety | Dry tons/acre | | | Mean |
| LCB 84X 16-66 | 11.56** | 12.28* | 13.75** | 12.53** |
| Ozark | 10.48* | 12.66** | 13.22* | 12.12* |
| Midland 99 | 10.32* | 10.12 | 12.63* | 11.02 |
| A-12245 | 9.85* | 10.82* | 11.54* | 10.74 |
| Tifton 44 | 10.15* | 10.25 | 11.69* | 10.69 |
| Vaughn's #1 | 8.99 | 9.22 | 8.89 | 9.03 |
| World Feeder | 8.70 | 7.87 | 8.82 | 8.46 |
| Scay Greenfield | 8.90 | 7.14 | 7.51 | 7.85 |
| Shrimplin | 5.71 | 6.27 | 7.65 | 6.54 |
| Mean | 9.41 | 9.63 | 10.63 | 9.89 |
| CV (%) | 15.05 | 16.77 | 18.20 | 16.89 |
| 5% LSD | 2.07 | 2.36 | 2.82 | 1.36 |

TABLE 3
Forage yields (tons dry matter/acre) of commercial and
experimental bermudagrass varieties in Test 98-1,
Eastern Research Station, Haskell, OK.

| | | | | | |
|---|---|---|---|---|---|
| | 1999 | 2000 | 2001 | 2002 | 4-Yr |
| Varieties | 4-Cuts | 4-Cuts | 3-Cuts | 4-Cuts | Mean |

Commercial Varieties - Available for Farm Use

| | | | | | |
|---|---|---|---|---|---|
| Midland 99 | 9.03* | 8.47 | 7.38** | 8.73 | 8.40* |
| Tifton 44 | 7.57 | 7.93 | 5.65 | 7.82 | 7.24 |
| Greenfield | 6.52 | 5.65 | 3.69 | 6.10 | 5.49 |

Experimental Varieties - Not Available for Farm Use

| | | | | | |
|---|---|---|---|---|---|
| ERS 94X 2-8 | 10.24** | 9.82** | 5.99 | 7.91 | 8.49** |
| LCB 84X 19-16 | 8.71 | 9.16* | 7.11* | 8.56 | 8.38* |
| ERS 94X 13-9 | 7.09 | 7.93 | 6.38 | 7.95 | 7.33 |
| LCB 84X 16-66 | 8.70 | 7.51 | 5.59 | 6.76 | 7.14 |
| ERS 94X 5-12 | 7.26 | 7.28 | 4.75 | 7.61 | 6.72 |
| ERS 94X 6-13 | 7.76 | 7.62 | 5.16 | 6.11 | 6.66 |
| SCRS-C | 6.83 | 7.17 | 5.79 | 7.66 | 5.40 |
| ERS-C | 5.78 | 5.93 | 3.43 | 6.46 | 5.40 |
| A12199 | 5.23 | 5.95 | 3.71 | 6.00 | 5.22 |
| Mean | 7.56 | 7.53 | 5.38 | 7.30 | 6.94 |
| CV(%) | 12.7 | 8.7 | 12.5 | 9.0 | 10.6 |
| 5% LSD | 1.38 | 0.94 | 0.97 | 0.94 | 0.51 |

**Highest numerical value in column
*Not significantly different from the highest numerical value in the column based on 5% LSD

TABLE 4
Forage yields (tons dry matter/acre) of commercial and experimental
bermudagrass varieties in Test 2001-1, Eastern Research Station,
Haskell, OK. 2002-2004.

| | Year | | | |
|---|---|---|---|---|
| | 2002 | 2003 | 2004 | |
| Variety | 4-cuts | 4-cuts | 3-cuts | Mean |

Commercial Varieties - Available for Farm Use

| | | | | |
|---|---|---|---|---|
| Ozark | 11.71** | 10.80* | 10.62** | 11.04** |
| Midland 99 | 11.49* | 10.32 | 10.54* | 10.78* |
| Tifton 44 | 10.90* | 10.22 | 10.35* | 10.49 |

Experimental Varieties - Not Available for Farm Use

| | | | | |
|---|---|---|---|---|
| A-12245 | 11.44* | 11.44** | 9.97* | 10.95* |
| ERS16S-4 | 10.97* | 11.10* | 9.61 | 10.56* |
| ERS16S-10 | 11.31* | 9.84 | 9.65 | 10.27 |
| A-12246 | 10.51 | 9.38 | 10.51* | 10.13 |
| ERS16S-2 | 10.61 | 10.23 | 9.37 | 10.07 |
| ERS16S-3 | 9.89 | 9.44 | 9.90 | 9.74 |
| ERS16S-7 | 10.89 | 9.06 | 9.23 | 9.73 |
| ERS16S-9 | 10.62 | 9.97 | 8.45 | 9.68 |
| ERS16S-6 | 10.20 | 9.15 | 9.12 | 9.49 |
| ERS16S-1 | 9.73 | 9.10 | 8.79 | 9.21 |
| ERS16S-8 | 9.29 | 8.48 | 9.45 | 9.08 |
| LCB 84X 16-66 | 9.61 | 9.68 | 7.70 | 8.99 |
| A-12244 | 9.03 | 7.53 | 9.32 | 8.63 |
| ERS16S-5 | 8.13 | 8.01 | 8.18 | 8.11 |
| Mean | 10.37 | 9.63 | 9.46 | 9.82 |
| CV (%) | 6.89 | 5.95 | 5.17 | 6.10 |
| 5% LSD | 1.02 | 0.81 | 0.69 | 0.48 |

**Highest numerical value in column
*Not significantly different from the highest numerical value in the column based on 5% LSD

TABLE 5
Forage yields (tons dry matter/acre) of commercial and experimental
bermudagrass varieties in Test 98-2, South Central Research Station,
Chickasha, OK. 1999-2002.

| | Harvest Year | | | | |
|---|---|---|---|---|---|
| | 1999 | 2000 | 2001 | 2002 | 4-Yr |
| Variety | 4-Cuts | 4-Cuts | 4-Cuts | 4-Cuts | Mean |

Commercial Varieties - Available for Farm Use

| | | | | | |
|---|---|---|---|---|---|
| Midland 99 | 13.11* | 10.97* | 9.28** | 9.14** | 10.62** |
| Tifton 44 | 12.03 | 12.26** | 7.65 | 8.45* | 10.10* |
| Greenfield | 8.91 | 6.73 | 4.04 | 5.52 | 6..30 |

Experimental Varieties - Not Available for Farm Use

| | | | | | |
|---|---|---|---|---|---|
| ERS 94X 2-8 | 14.21** | 10.31* | 8.68* | 6.80 | 10.00* |
| LCB 84X 19-16 | 11.72 | 10.10* | 8.41* | 7.92 | 9.54 |
| ERS 94X 13-9 | 10.95 | 10.36* | 8.53* | 8.02 | 9.46 |
| SCRS-C | 11.69 | 10.40* | 7.35 | 7.26 | 9.17 |
| LCB 84X 16-66 | 13.00* | 7.46 | 6.08 | 6.82 | 8.34 |
| ERS 94X 6-13 | 10.43 | 8.90 | 6.20 | 4.99 | 7.63 |
| A12199 | 8.56 | 7.21 | 6.03 | 6.15 | 6.98 |
| ERS 94X 5-12 | 9.01 | 6.82 | 5.72 | 6.02 | 6.89 |
| ERS-C | 8.81 | 4.98 | 3.32 | 4.32 | 5.36 |
| Mean | 11.03 | 8.87 | 6.77 | 6.78 | 8.37 |
| CV (%) | 12.9 | 18.0 | 14.3 | 18.3 | 15.7 |
| 5% LSD | 2.05 | 2.30 | 1.39 | 1.79 | 0.92 |

**Highest numerical value in column
*Not significantly different from the highest numerical value in the column based on 5%

TABLE 6
Forage yields (tons dry matter/acre) of commercial and experimental
bermudagrass varieties in Test 2001-2, South Central Research
Station, Chickasha, OK. 2002-2005.

| | Harvest Year | | | | | |
|---|---|---|---|---|---|---|
| | 2002 | 2003 | 2004 | 2005 | Mean¹ | |
| | 4 cuts | 4 cuts | 3 cuts | 2 cuts | 2002-05 | 2003-05 |
| Variety | Tons DM/acre | | | | | |
| Midland | 10.97* | 10.91* | 10.77** | 7.04* | 9.92* | 9.57** |
| 99 | | | | | | |
| Ozark¹ | — | 10.36* | 10.06* | 7.35** | — | 9.26* |
| Tifton | 11.72* | 10.19* | 9.78* | 5.17 | 9.26 | 8.45 |
| 44 | | | | | | |
| A12246 | 12.04** | 10.95* | 10.74* | 6.50* | 10.06** | 9.40* |
| A12245 | 11.82* | 11.05* | 10.15* | 5.61 | 9.66* | 8.94* |
| ERS16S | 11.44* | 11.22** | 9.54* | 5.92 | 9.53* | 8.89* |
| 03 | | | | | | |
| ERS16S | 12.44* | 10.14* | 9.20 | 5.98 | 9.44 | 8.44 |
| 04 | | | | | | |
| ERS16S | 9.98 | 9.67 | 9.10 | 6.18 | 8.73 | 8.31 |
| 08 | | | | | | |
| ERS16S | 10.37 | 9.24 | 9.08 | 4.74 | 8.36 | 7.69 |
| 01 | | | | | | |
| LCB | 10.08 | 8.74 | 8.91 | 4.80 | 8.13 | 7.48 |
| 84X16- | | | | | | |
| 66 | | | | | | |
| A12244 | 9.73 | 8.30 | 7.86 | 4.56 | 7.61 | 6.91 |
| ERS16S | 9.79 | 9.05 | 7.47 | 3.89 | 7.55 | 6.80 |
| 05 | | | | | | |
| Mean | 10.94 | 9.98 | 9.40 | 5.64 | 8.93 | 8.34 |
| 5% | 1.30 | 1.49 | 1.25 | 1.01 | 0.59 | 0.71 |
| LSD | | | | | | |
| CV (%) | 8.21 | 10.36 | 9.22 | 12.45 | 9.92 | 10.52 |

¹Ozark yields were not measured in 2002 due to herbicide injury. Ozark plots had recovered by 2003. Plots received 300 lbs. N/acre/yr applied in three equal applications

TABLE 7
Forage yields of bermudagrass varieties and experimental lines at
Kansas State University Southeast Agricultural Research Center,
Mound Valley, KS. 1993-1995.

| | Year | | | |
|---|---|---|---|---|
| | 1993 | 1994 | 1995 | 3-Yr |
| Entry | Tons/acre @ 12% moisture | | | Mean |
| 74X 11-2 | 5.91 | 9.54 | 7.81 | 7.75 |
| LCB 84X 16-66 | 6.31 | 8.18 | 6.28 | 6.92 |
| LCB 84X 19-16 | 6.05 | 8.36 | 6.04 | 6.82 |
| Ozark | 4.60 | 8.10 | 7.73 | 6.69 |
| LCB 84X 15-49 | 6.04 | 8.01 | 5.92 | 6.65 |
| Hardie | 5.97 | 7.85 | 6.04 | 6.62 |
| 74X 12-12 | 6.12 | 7.22 | 6.52 | 6.62 |
| LCB 84X 9-45 | 6.38 | 7.52 | 5.70 | 6.53 |
| LCB 84X 19-31 | 5.74 | 7.41 | 5.96 | 6.37 |
| Tifton 44 | 5.23 | 7.05 | 5.79 | 6.02 |
| LCB 84X 14-31 | 5.88 | 6.99 | 4.58 | 5.82 |
| LCB 84X 19-23 | 5.54 | 6.52 | 4.75 | 5.60 |
| LCB 84X 12-28 | 5.94 | 6.04 | 4.22 | 5.40 |
| LCB 84X 15-26 | 5.10 | 5.16 | 5.18 | 5.14 |
| Midland | 4.40 | 5.88 | 4.86 | 5.05 |
| LCB 84X 21-57 | 5.24 | 4.25 | 4.74 | 4.74 |
| LCB 84X 18-62 | 4.62 | 4.88 | 4.43 | 4.64 |
| Greenfield | 4.54 | 4.28 | 4.81 | 4.54 |
| World Feeder | 4.11 | 4.30 | 4.44 | 4.28 |
| LCB 84X 16-55 | 4.28 | 3.86 | 3.88 | 4.00 |
| Mean | 5.40 | 6.57 | 5.45 | 5.81 |
| 5% LSD | 0.59 | 0.79 | 0.82 | |

TABLE 8
Mean neutral detergent fiber (NDF), acid detergent fiber (ADF),
acid detergent lignin (ADL), crude protein (CP), and in vitro
digestible dry matter (IVDMD) of Midland, Tifton 44, and
LCB 84X 16-66 bermudagrass cultivars. Stillwater, OK, 1986-87.

| | Quality trait | | | | |
|---|---|---|---|---|---|
| | NDF | ADF | ADL | CP | IVDMD |
| Cultivar | g kg⁻¹ | | | | |
| LCB 84X 16- | 720 | 342 | 38 | 135 | 604 |
| 66 | | | | | |
| Tifton 44 | 762 | 334 | 38 | 121 | 553 |
| Midland | 725 | 331 | 40 | 138 | 553 |

Values are means of six sampling dates (Jun. 17, 1986, Jul. 17, 1986, Aug. 22, 1986, Jun. 25, 1987, Aug. 19, 1987, Oct. 5, 1987). Plots were unreplicated.

We claim:

1. A bermudagrass plant substantially as described and illustrated in the specification herein.
